(19) United States
(12) Patent Application Publication
(10) Pub. No.: US 2026/0263653 A1
(43) Pub. Date: Sep. 10, 2026

(54) DYNAMIC SUTURE FILAMENTS

(71) Applicant: RLFA Biomedical Innovations Ltd, Dublin (IE)

(72) Inventors: Robert FLAVIN, Dublin (IE); Olivia WEST, Dublin (IE)

(21) Appl. No.: 19/557,589

(22) Filed: Mar. 5, 2026

(30) Foreign Application Priority Data
Mar. 5, 2025 (GB) 2503214.5

(51) Int. Cl. A61L 17/10; A61L 17/08; A61L 17/14; C08L 89/06

(52) U.S. Cl. CPC A61L 17/105; A61L 17/08; A61L 17/145; C08L 89/06

(57) ABSTRACT

A suture filament comprising a first material and a second material. The first material is absorbable, and the second material is non-absorbable. The first material, second and third materials are bonded together to define a single composite filament. Also provided are suture and mesh arrangements comprising one or more of the suture filaments.

### CROSS-REFERENCE TO RELATED APPLICATIONS

This application claims priority to United Kingdom Patent Application No. 2503214.5 filed on Mar. 5, 2025, which is incorporated by reference herein.

### FIELD

The specification relates to the field of sutures for use in medical applications.

### BACKGROUND

Sutures are commonly made of either absorbable material or non-absorbable material. US 2009/0143819 describes a suture comprising at least one filament to which a coating is applied to enable the suture to be easily inserted into tissue. The coating is of a different material to the suture. In this case the coating only applied after the filament is formed is of an absorbable material. Thus in this arrangement a filament is simply a coated filament the coating does not affect or contribute to the structure of the filament.

The arrangements of the present specification are directed to providing improved and alternative arrangements of suture filaments for use in surgical applications including for example in orthopedic applications.

### SUMMARY

The scope of the disclosure is in accordance with the appended claims.

In a first embodiment, there is provided a suture filament (100, 200, 300), as defined in claim 1.

There is provided a suture filament comprising a first material (12, 42); a second material (22, 62); and a third material (32, 72); wherein the first material (12, 42) is absorbable, the second material (22, 62) is absorbable or non-absorbable; and the third material (32, 72) is absorbable or non-absorbable; wherein the materials are bonded together to define a single composite filament.

In contrast to previous suture filaments, the filament of the disclosure comprises in one embodiment first, second and third materials only. In other embodiments the filament comprises at least first second and third materials. The combination and proportions of the first, second and third materials are selected and controlled to define a dynamic suture filament that changes in form over time. How the suture filament changes overtime can be controlled by the selections of the materials. The absorbable materials may be selected and configured to absorb at different rates overtime. The non-absorbable components work together in synergy with the absorbable components to provide in combination the strength and forces required. The dynamic suture filament thus provides for changes in strength over time. For example, the strength may be controlled to be applied at 100% initially and for a first duration or time period of a first phase of healing. The strength applied may reduce gradually to 75% over a second time period. The strength applied may be maintained at 75% during a third phase for a third time period. The strength may then reduce further to 50% for a fourth time period, and so on. Thus the materials and the structural forms of the filaments according to the specification provides for controlled and dynamics changes in the strength of the filament and the resultant forces applied over time.

Further optional features are provided in accordance with the dependent claims.

In further embodiments, there are provided mono-filament sutures and multi-filament sutures as defined in claims 20-22 comprising one or more filaments according to the embodiments of the disclosure.

Further embodiments provide other structures comprises of one or more filaments according to the specification, for example a mesh structure as defined in claims 23 and 24.

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

In general, the disclosed suture comprises a filament that comprises a first material, a second material, and a third material wherein: the materials are bonded to one another, at least one material is absorbable, and the remaining materials are either absorbable or non-absorbable.

Other illustrative variations within the scope of the disclosure will become apparent from the detailed description provided hereinafter. The detailed description and enumerated variations, while disclosing optional variations, are intended for purposes of illustration only and are not intended to limit the scope of the disclosure.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1A illustrates an exemplary arrangement of a cross-section of a first filament of a first type according to an embodiment of the disclosure of the specification; FIG. 1B illustrates an exemplary arrangement of a cross-section of the suture filament of FIG. 1A further comprising a coating;

FIGS. 2A, 2B, 2C and 2D each illustrate exemplary arrangements of cross-sections of a suture filament of a second type according to an embodiment of the disclosure of the specification;

FIGS. 3A and 3B illustrate exemplary arrangements of a cross-section of a suture filament of a third type according to an embodiment of the disclosure of the specification;

FIG. 4 illustrates an exemplary arrangement of a cross-section of a muti-filament suture according to an embodiment of the disclosure of the specification;

FIG. 5A shows a perspective cut-away view of a suture filament of the exemplary arrangement of FIG. 1A; and FIG. 5B shows a perspective cut-away of a suture filament of the exemplary arrangement of FIG. 2B; and

FIG. 6 shows a block diagram of an exemplary method of manufacture of a filament according to the specification.

The drawings may be shown exaggerated in scale or in schematic in the interest of clarity and conciseness.

### DETAILED DESCRIPTION

The specific details of the single embodiment or variety of embodiments described herein are to the described product or methods of use. Any specific details of the embodiments are used for demonstration purposes only and no unnecessary limitations or inferences are to be understood from there.

It is noted that the embodiments reside primarily in combinations of components and procedures related to the products. Accordingly, the product and components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

Musculoskeletal soft tissue repair has evolved over the decades from natural non-absorbable sutures to synthetic non-absorbable sutures and to absorbable sutures. With this evolution, sutures of varying diameters were developed and the tensile strength of sutures improved relative to their diameter size. With the advent of polymer-based suture material, the handling and potential soft tissue irritation issues became apparent. Knot tying irritation issues and knot prominence irritation issues became the biggest disadvantages of these polymer-based sutures and the move from monofilament to braided sutures addressed the knot tying irritation issues while the knot prominence irritation issues were exacerbated.

Furthermore, often as a result of musculoskeletal soft tissue repair, the capabilities of the anatomy affected by the repair(s) are heavily altered. For example, replacing or repairing a tendon or ligament with a permanent suture replaces the range of motion, elasticity, and flexibility of the tendon or ligament with the relative elasticity, flexibility and range of motion of the mechanical properties of the suture material. This results in patients experiencing a permanent loss of their normal bodily functions in the repaired area.

In general, the exemplary arrangements and embodiment disclosed herein are directed to addressing these and other issues.

According to the embodiments and exemplary arrangements of the specification there is provided a suture filament 100, 200, 300. The suture filaments 100, 200, 300 comprise: a first material, a second material, a third material. The first material is absorbable, and the second and third materials may be absorbable or non-absorbable. According to the embodiments and exemplary arrangements of the specification various structural configurations 101, 201, 301 of embodiments according to the different types of suture filament 100, 200 and 300 are also described. In a preferred arrangement, the first material is absorbable, and the second is non-absorbable and the third material is absorbable or non-absorbable.

The first, second and third materials are bonded to create a single composite filament.

In a preferred arrangements the filaments comprise the first, second and third materials only. It is however appreciated that in other arrangements, the suture filament may further comprise other additional materials. In an exemplary arrangement comprising other additional materials, these may be absorbable or non-absorbable. These additional materials when provided as part of the composite filament are also bonded to one or more of the first, second and third materials to create a single composite filament.

In some embodiments, sutures according to the exemplary arrangements described can include at least three materials. In one arrangement a suture comprises a single suture filament according to the specification. The single suture filament comprising a first, second, third materials only. In other arrangements, the single suture filament may optionally comprise further additional number of materials.

In a further exemplary arrangement, a suture may be provided comprising a suture filament of the specification comprising a first, second and third materials in combination with another material. Sutures provided according to the exemplary arrangements may comprise a single filament suture of the specification. Sutures provided according to the exemplary arrangements may alternatively comprises multiple suture filaments.

In some embodiments, a first material of the at least three materials of a filament according to the specification can be absorbable and a second material of the at least three materials can be non-absorbable and a third material of the at least three materials can be non-absorbable. In some embodiments, the first material of the at least three materials is non-absorbable and the second material of the at least three materials is absorbable and the third material of the at least three materials can be absorbable. In some embodiments, a first material of the at least three materials can be non-absorbable, and a second material of the at least three materials can be absorbable, and any additional number of materials can be absorbable or non-absorbable.

Suture size and strength are common characteristics used for selecting an appropriate suture for surgical procedures. In some embodiments, since at least one of the at least three materials of the suture can be absorbable, the size (e.g., width and/or diameter) of the suture can decrease over time, and for example, can decrease while a sutured wound heals. When the size of the suture decreases, natural tissue can re-enter the wounded site more quickly, allowing the wounded site to heal faster and allowing the body to repopulate the evacuated area with its natural cells. With this faster healing and return of natural cells, the body may regain some of its original performance capabilities at the affected site, rather than relying on the capabilities of the materials used in the repair(s).

Since the non-absorbable material(s) of the suture are not absorbed, the strength of the suture is maintained over time while the absorption of the absorbable material(s) allows the natural tissue of the body to regrow and re-enter the wounded site. As the wounded site begins to heal, the required strength of the suture decreases, which prevents the suture from breaking as its size decreases. The suture can remain sufficiently strong for even orthopedic applications. When the size of the suture decreases, the amount of space in the wounded site that is occupied by the suture also decreases, which reduces patient discomfort. Natural bodily processes can begin to repair the wounded site and, with a smaller suture, more of the natural tissue can be restored.

In general, the embodiments disclosed herein relate to a suture that can be used to perform any musculoskeletal soft tissue repair. For example, the suture can be used to perform a tendon-to-tendon repair, a ligament-to-ligament repair, a tendon-to-bone repair, a ligament-to-bone repair, a fascial tissue repair, a cutaneous repair, a subcutaneous repair, or any combination thereof. In some embodiments, the suture can be of any desired length suitable for performing any musculoskeletal soft tissue repair.

In some embodiments, the suture filament is formed by thermally bonding the at least three materials into a single composite filament. In some embodiments, the at least three materials can be thermally bonded. In some embodiments, forming the filaments includes spinning the at least three materials. Spinning the at least three materials can include melt spinning the at least three materials, electrospinning the at least three materials, or solution spinning the at least three materials. In some embodiments, melt spinning the at least three materials can comprise extruding the at least three materials. The at least three materials can be extruded by an extruder. In some embodiments, melt spinning the at least three materials can include heating or melting the at least three materials and passing the at least three materials through, for example, a spinneret. In some embodiments, forming the filament can further comprise drawing the at least three materials to elongate the at least three materials.

In some embodiments, a material is absorbable if the material can be absorbed by the human body over time, and for example, within approximately 60 days to 180 days.

In some embodiments, the at least three materials can be bonded to form a filament with any desired cross-section. In some embodiments, cross-sections at different positions along a length of the filament can generally be the same.

In some embodiments, the filament may comprise a core-sheath-sheath suture filament. For example, the filament can include an elongate core made primarily of the first material, wherein the elongate core is bonded to a sheath made primarily of the second material and the sheath made primarily of the second material is bonded to a sheath made primarily of the third material. In some embodiments, the elongate core can be positioned along a center axis of the sheath. In some embodiments, a center axis of the elongate core can be aligned with a center axis of the sheath(s). In some embodiments, the elongate core is not positioned along the center axis of the sheath(s). In some embodiments, the center axis of the elongate core is not aligned with the center axis of the sheath(s). In some embodiments, the ratio of the elongate core to the bonded sheath(s) can be as small 20:80. In some embodiments, ratio of the elongate core bonded to the sheath(s) can be as large as 80:20.

In some embodiments, the filament can be an ‘islands-in-the-sea’ suture filament. For example, the filament can have a cross-section with a plurality of separate regions made of the second material and third material, wherein the separate regions are surrounded by and inside a region made of the first material. In some embodiments, the final filament also referred to as the final single composite filament can include a plurality of regions made primarily of the second material and third material, wherein the plurality of regions of the second material and third material are surrounded by a common sheath made primarily of the first material. The regions of the second material and third material may be considered as strands for example of cylindrical form, as the regions of the second material and third material have an elongated form and extend within the filament structure parallel to the longitudinal axis (X-direction) of the final filament.

In some embodiments, each filament in a plurality of filaments can be separated from any other filament in the plurality of filaments by a predetermined distance.

In some embodiments, in a filament the ratio of the first material to the second material and third material can be as small as 20:80. In some embodiments, the ratio of the first material to the second material and third material can be as large as 80:20.

In some embodiments the ratios by volume of the proportions of the first, second and third materials may be in the ranges of 60:20:20, 20:60:20 and 20:20:60.

In other embodiment the ratios of first material to the second material and third materials are adjustable within the ranges of 80:10:10, 10:80:10, and 10:10:80; and 70:15:15, 15:70:25, and 15:15:70; and 60:20:20, 20:60:20 and 20:20:60.

The different materials may be selected as required with the above ratios of the proportions of the first, second and third materials-to provide a filament 100, 200, 300 and sutures 1000, 1001 and 2000 that are configured to provide strength that controllably varies with time. For example over intervals of time the strength may be varied from an initial 100% strength to 75% strength to 50% strength etc—as required taking account of the particular surgery and as required to support healing over time. For example when deployed the filament(s) have a 100% strength which is decreased over time. The filaments by selection of the materials and form is thus configured to provide a front-loaded maximum strength.

In some embodiments, the filament can be a ‘segmented pie’ suture. For example, the filament can have a cross-section that includes a first pie-shaped region made primarily of the first material, the cross-section further including a second pie-shaped region made primarily of the second material and a third pie-shaped region made primarily out of the third material. In some embodiments, the first pie shaped region can have a first side extending along a first radius of the cross-section, a second side extending along a second radius of the cross-section, and a third side extending along an arc connecting the first side to the second side. In some embodiments, a first angle can be formed between the first side and the second side of the first pie-shaped region. In some embodiments, the second pie shaped region can have a fourth side extending along the first radius of the cross-section, a fifth side extending along the second radius of the cross-section, and a sixth side extending along a second arc connecting the fourth side to the fifth side. In some embodiments, a second angle can be formed between the fifth side and the sixth side of the second pie-shaped region.

In some embodiments, the first pie-shaped region, the second pie-shaped region and the third pie-shaped region can have any suitable sizes. In some embodiments, the first pie-shaped region can be larger than the second pie-shaped region and the third pie-shaped region. In some embodiments, the second pie-shaped region can be larger than the first pie-shaped region and the third pie-shaped region. In some embodiments the third pie-shaped region can be larger than the first pie-shaped region and the second pie-shaped region. In some embodiments, the first pie-shaped region, the second pie-shaped region and the third pie-shaped region can be the same size. In some embodiments, the cross-section of the suture filament can include one or more additional pie-shaped regions made primarily of different materials and having any suitable size(s).

In some embodiments, the filament can be treated as a monofilament suture. Overall the suture filaments according to the exemplary arrangements of the specification are described as self-contained monofilaments. The at least three materials are provided in a single monofilament structure

In some embodiments, multiple strands of the filament can be braided to form a multifilament suture

In some embodiments, the filament can be used as an elongate core around which to braid a multifilament suture.

In some embodiments, the filament can be braided around an elongate core to form a multifilament suture.

In some embodiments, two or more sutures can be made. In some embodiments, the two or more sutures can be braided to form a multifilament suture. The multifilament suture can have a first filament made primarily of a first suture, a second filament made primarily of a second suture, and any number of filaments made primarily of any number of materials.

In some embodiments, the suture and an implantable material can be braided to form a braided suture. In some embodiments, the suture and a filament made of the implantable material can be braided to form a braided suture.

In some embodiments, multiple strands of the filament can be twisted, braided, woven, knitted or chemically bonded to form a suture mesh.

In some embodiments, the filament can be twisted, braided, woven, knitted or chemically bonded with additional material(s) to form a suture mesh.

In some embodiments, non-absorbable materials can include, or be selected from the following: polyester, polyethylene terephthalate (PET), ultra-high molecular weight polyethylene (UHMWPE), nylon, or any combination thereof.

In some embodiments, absorbable materials can include or be selected from the following: polyglactin, dexon, poliglecaprone, polyglycolic acid (PGA), polydioxanone (PDO), poly-L-lactic 25 acid (PLLA), or any comparable polymer or any combination thereof.

In some embodiments, the suture can have a size ranging from approximately 5/0 AWG or 5/0 SWG to approximately 5 AWG or 5 SWG.

In some embodiments, two or more filaments can be braided, wrapped over one another, twisted over one another, sheathed by any material, etc. to form a multifilament suture. The multifilament suture can have any picks and/or twists per inch and any material composite ratio of braided, sheathed, cored, etc. strands resulting in suture sizes 5/0 to #5. The multifilament suture can have a size ranging from approximately 5/0 AWG or 5/0 SWG to approximately 5 AWG or 5 SWG.

In some embodiments, the suture or mesh can be attached to a suture anchor.

In some embodiments, the suture or mesh can be attached to a needle.

In some embodiments, the suture or mesh can be elongate.

The following description of variants is only illustrative of components, elements, acts, products, and methods considered to be within the scope of the disclosure and are not in any way intended to limit such scope by what is specifically disclosed or not expressly set forth. The components, elements, acts, products, and methods as described herein may be combined and rearranged other than as expressly described herein and are still considered to be within the scope of the disclosure.

According to variation 1, a suture can include a filament that includes a first material, a second material and a third material, wherein the materials are bonded; the first material is absorbable; the second material is non-absorbable; and the third material is non-absorbable.

According to variation 2, a suture can include a filament that includes a first, a second material and a third material, wherein the materials are bonded; the first material is absorbable; the second material is absorbable; and the third material is non-absorbable.

Variation 3 can include the suture as in variation 1, wherein the first material is selected from the following or includes one or more of the following: polyglactin, dexon, poliglecaprone, polyglycolic acid (PGA), polydioxanone (PDO), poly-L-lactic acid (PLLA), or any comparable polymer or any combination thereof.

Variation 3 can include the suture as in variation 1, wherein the second material and third material are selected from the following or includes one or more of the following polyester, polyethylene terephthalate (PET), ultra-high molecular weight polyethylene (UHMWPE), nylon, or any combination thereof.

Variation 4 can include the suture as in variation 2, wherein the first material is selected from the following or includes one or more of the following polyester, polyethylene terephthalate (PET), ultra-high molecular weight polyethylene (UHMWPE), nylon, or any combination thereof.

Variation 4 can include the suture as in variation 2, wherein the second material and third material are selected from the following or includes one or more of the following: polyglactin, dexon, poliglecaprone, polyglycolic acid (PGA), polydioxanone (PDO), poly-L-lactic acid (PLLA), or any comparable polymer or any combination thereof.

The materials of the filament may be selected depending on applications and intended use. For example, the absorbable material(s) may be chosen based on its total degradation time. For example, when repairing a ligament a suture requires a higher initial strength than when repairing a tendon. In the case of ligament repair, the absorbable material(s) would be chosen for a slower rate of degradation and resorption, allowing the initial strength to remain for a longer period of time. Furthermore, two absorbable materials may be chosen for different degradation times, creating a staged degradation and maintaining three phases of strength rather than two.

Further the ratio of the absorbable material to the non-absorbable material within the filament structure may vary as required depending on applications and intended use. The ratio may for example be determined based on total degradation time of the absorbable material(s). For example, when repairing a ligament a suture requires a higher initial strength than when repairing a tendon. In the case of ligament repair, the ratio of the absorbable material to the nonabsorbable material would be lesser than the ratio of the absorbable material to the nonabsorbable material in a suture used for tendon repair, allowing more of the non-absorbable material to remain at the repair site permanently.

Variation 5 can include the suture as in variation 1 or variation 2, wherein the suture includes an elongate core made primarily of the first material, wherein the elongate core is bonded to a sheath made primarily of the second material and the elongate sheath of the second material is bonded to a sheath made primarily of the third material.

According to variation 6, a suture can include a first filament that includes a first material, wherein the first material is absorbable; a second filament that includes a second material, wherein the second material is non-absorbable; and a third filament that includes a third material wherein the third material is absorbable or non-absorbable; wherein the first filament, the second filament and the third filament are braided.

According to variation 7, a suture can include a first filament that includes a first material a second material and a third material wherein: the materials are bonded; the first material is absorbable; the second material is non-absorbable; and the third material is absorbable or non-absorbable; a second filament that includes a fourth material, a fifth material, and a sixth material wherein: the materials are bonded; the fourth material is absorbable; the fifth material is non-absorbable; and the sixth material is absorbable or non-absorbable; wherein the first filament 20 and the second filament are braided.

According to a variation 8 a multifilament suture may be provided, comprising: A first filament comprising a first, second and third material and at least one other filament comprising a first, second and third material, twisted, braided or otherwise mechanically combined to 25 form said multifilament suture, wherein the first and at least one other filament are the same.

The first material being absorbable; the second material being non-absorbable; and the third material being absorbable or non-absorbable.

According to a variation 9 a multifilament suture may be provided, comprising: at least one filament comprising a first, second and third material, the first material being absorbable; the second material is non-absorbable; the third material is absorbable or non-absorbable, twisted, braided or otherwise mechanically combined with at least one other filament of a second different type, wherein the filament of the second different type is comprised of materials different from the materials of the filament of the first type.

According to a variation 10 a multifilament suture may be provided, comprising: a first filament comprising a first, second and third material, wherein the materials are bonded to form a single composite filament; and wherein the first material is absorbable; the second material is non-absorbable; and the third material is absorbable or non-absorbable; and a second filament comprising a fourth, fifth and sixth material, wherein the fourth, fifth and sixth materials are bonded to form a single composite filament, and wherein the fourth material is absorbable; the fifth material is non-absorbable; and the sixth material is absorbable or non-absorbable wherein the first filament and the second filament are braided together.

According to a variation 11 a suture mesh may be provided, comprising: a first filament and one or more other filaments, the first filament comprising a first, second and third material wherein the first materials are bonded to form a single composite filament; and wherein the first material is absorbable; the second material is nonabsorbable; and the third material is absorbable or non-absorbable; wherein the first filament is woven, knit, or chemically bonded to at least one other filament, wherein the first and at least one other filament are the same.

According to a variation 12 a suture mesh may be provided, comprising: a first filament, the first filament comprising a first, second and third material, wherein the materials are bonded to form a single composite filament; and wherein the first material is absorbable; the second material is non-absorbable; and the third material is absorbable or non-absorbable; wherein the first filament is woven, knit or chemically bonded to one or more other filaments of different materials.

The specification is aimed at providing improved suture filaments 100, 200, 300 according to various embodiments. The suture filaments 100, 200 and 300 comprise first, second and third materials. The different filaments 100, 200 and 300 comprise the first, second and third materials in different exemplary structural arrangements.

These structural arrangements are described further below with reference to the drawings. In the following, the materials are described in further detail.

The suture filaments 100, 200, 300 can effectively be considered as dynamic or smart suture filaments, on the basis that the process of manufacture and the composition and form of the filaments provide dynamic filaments that take account of healing over time and changing requirements for suture strength across healing phases.

The materials of the suture filaments 100, 200, 300 are key features of the disclosure. The sutures are comprised of the first, second and third materials. The selection of the materials, the proportions of the materials, and parameters and form of the filaments and sutures all combine to define a particular dynamic suture according to the disclosure. The filaments are configured to degrade and change dynamically over time, in particular the forms and structures of the filaments are configured to change dynamically over time. The rate of change is controllable by the selection of the materials of the suture and the form of the suture.

The combination and proportions of the first, second and third materials are selected and controlled to define a dynamic suture filament that changes in form over time. How the suture filament changes overtime can be controlled by the selections of the materials. The absorbable materials may be selected and configured to absorb at different rates overtime. The non-absorbable components work together in synergy with the absorbable components to provide in combination the strength and forces required. The dynamic suture filament thus provides for changes in strength over time. For example, the strength may be controlled to be applied at 100% initially and for a first duration or time period of a first phase of healing. The strength applied may reduce gradually to 75% over a second time period. The strength applied may be maintained at 75% during a third phase for a third time period. The strength may then reduce further to 50% for a fourth time period, and so on. Thus the materials and the structural forms of the filaments according to the specification provides for controlled and dynamic changes in the strength of the filament and the resultant forces applied over time.

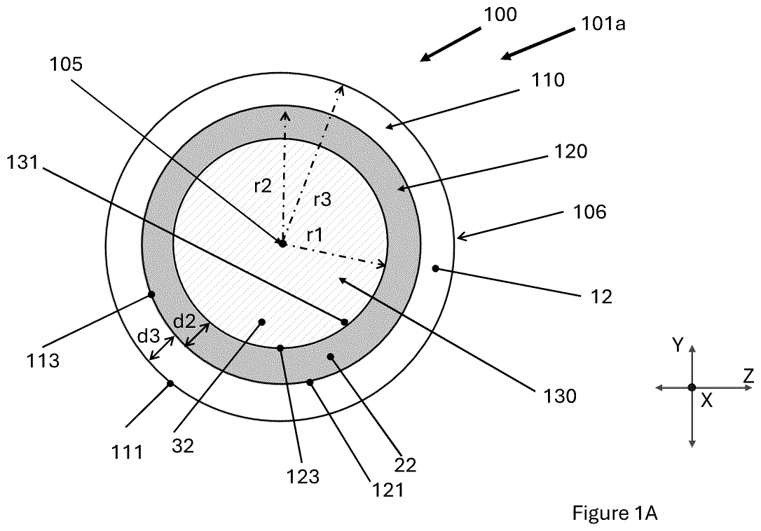

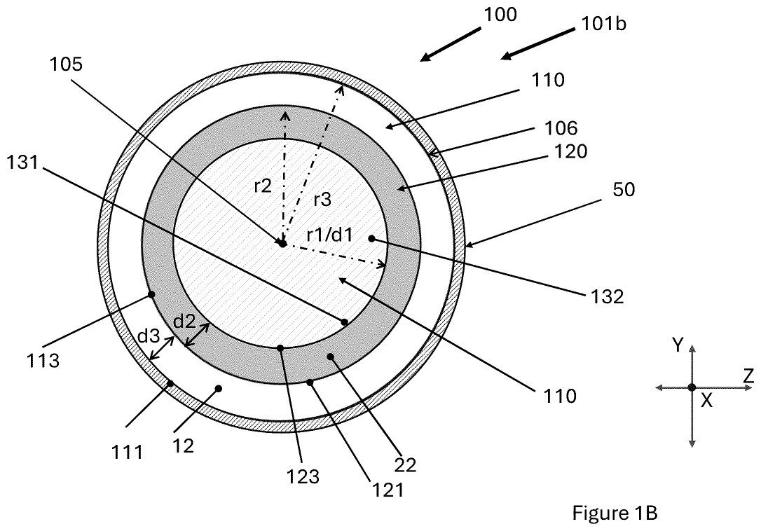

Referring to FIGS. 1, 2 and 3, exemplary filaments 100, 200 and 300 comprising first 12, second 22 and third 32 materials are shown. The first material 12 is absorbable. The second material 22 is non-absorbable. The third material 32 is absorbable 32A or non-absorbable 32N.

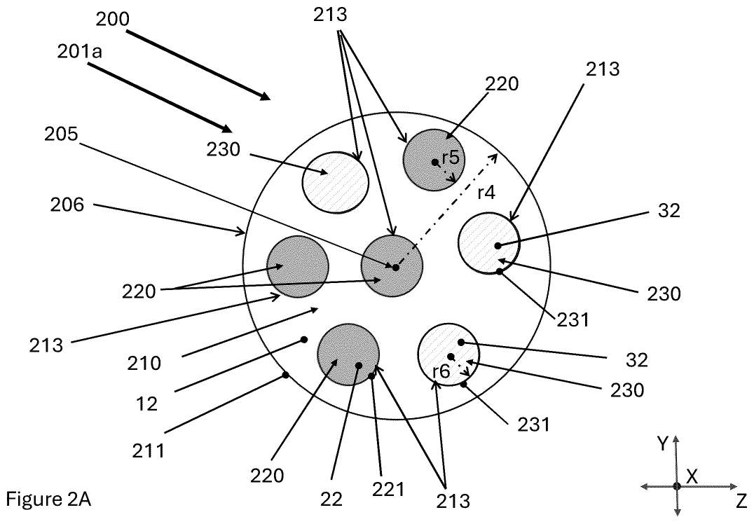

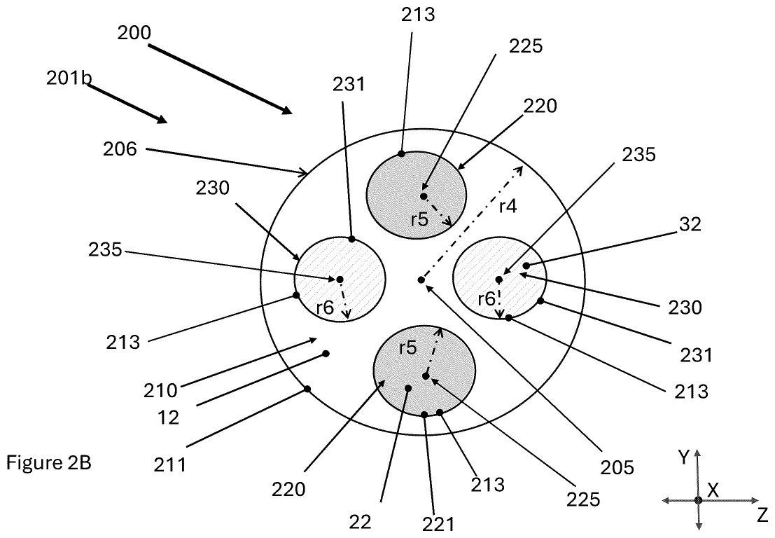

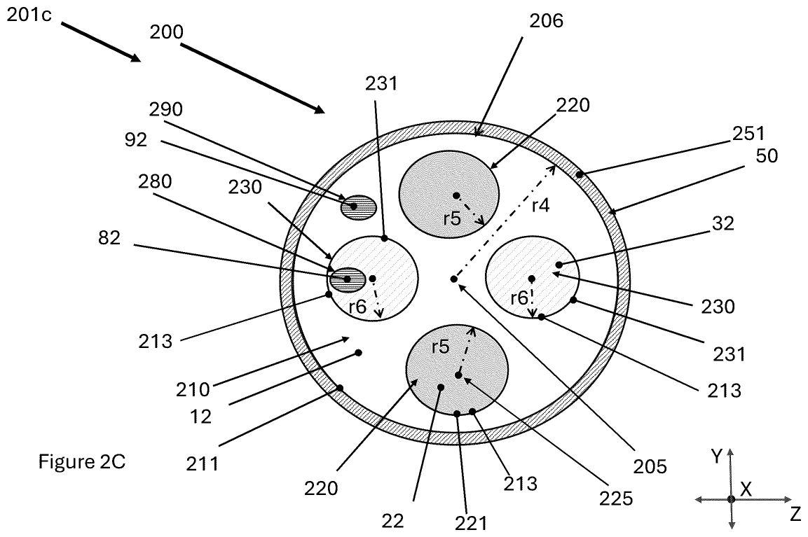

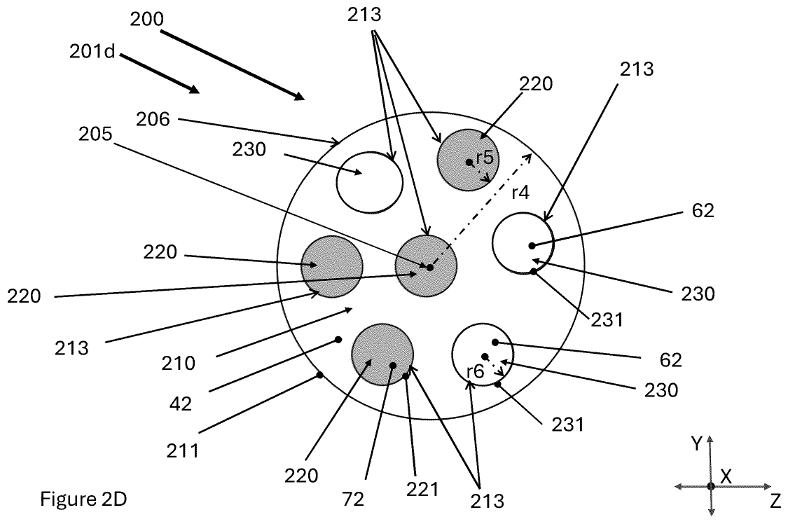

To illustrate further how different filaments of different materials may be used to form a dynamic suture, a further exemplary arrangement is described with reference to FIG. 2D in which a filament 200 in a configuration 201d comprises fourth, fifth and sixth materials. The fourth material 42 is absorbable. The fifth material 62 is non-absorbable. The sixth material 72 is absorbable 72A or non-absorbable 72N. This alternative arrangement 201d may be combined with a filament 100, 200, 300 comprising first, second and third materials to form a multi-filament suture 1001.

The non-absorbable materials 22, 32A, 62, 72N may comprise, include, or be selected from the following: polyester, polyethylene terephthalate (PET), ultra-high molecular weight polyethylene (UHMWPE), nylon, or any combination thereof.

The absorbable materials 12, 32A, 42, 72A may comprise, include or be selected from the following: polyglactin, dexon, poliglecaprone, polyglycolic acid (PGA), polydioxanone (PDO), poly-L-lactic 25 acid (PLLA), or any comparable polymer or any combination thereof.

The specification provides sutures 1000 that are comprised of single filaments 100, 200, 300. The specification further provides sutures 1001 that are comprised of multiple filaments. The sutures can have a size ranging from approximately 5/0 AWG or 5/0 SWG to approximately 5 AWG or 5 SWG.

To form a multiple filament suture 1001 two or more filaments 100, 200, 300 can be braided, wrapped over one another, twisted over one another, sheathed by any material, etc. to form a multifilament suture. The multifilament suture 1001 can have any picks and/or twists per inch and any material composite ratio of braided, sheathed, cored, etc. strands resulting in suture sizes 5/0 to #5. The multifilament suture 1001 can have a size ranging from approximately 5/0 AWG or 5/0 SWG to approximately 5 AWG or 5 SWG.

The disclosure in relation to the materials above applies to filaments according to each of the embodiments described below. The embodiments below provide examples of how the various materials may be combined in different filament types having different basic structural forms. The different structural forms and arrangements described allow for definition of how the different materials are arranged within the composite filament, according to the exemplary arrangements. It will be appreciated that the form of components shown in an example cross section as circular may in the final composite film have a different final form. While the forms may be different, the proportions of the different materials and their relative location will be broadly similar. The description of the components is provided to highlight examples of how the materials may be arranged in the different proportions internally within a filament.

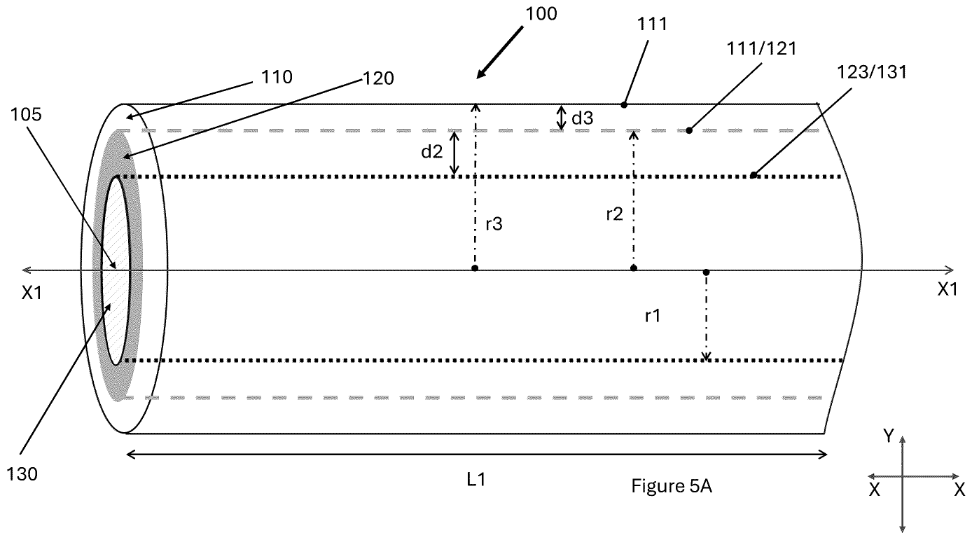

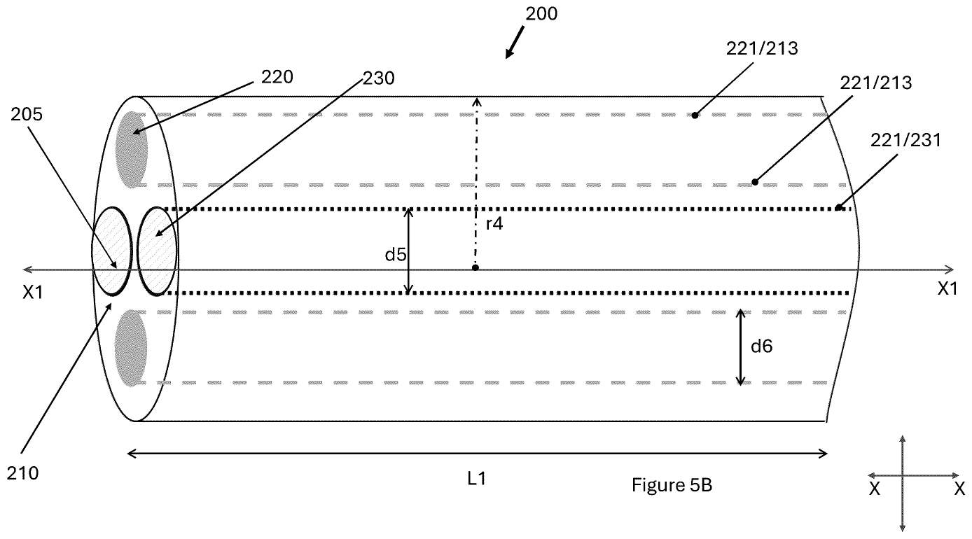

Following on from the above description, a filament 100 of a first type according to an exemplary arrangement of the specification is described with reference to FIG. 1A and FIG. 5A. As described above the filament 100 comprises a composite filament or monofilament. The first type of filament 100 is a core-sheath-sheath filament.

In the specification the terms component, region, portion and strand have been used interchangeably to refer to the different structural features or components of a filament.

FIG. 1 shows a lateral cross-sectional view of the filament 100 comprising a first material 12 and a second material 22, and a third material 32. The filament 100 comprises a first component 110 of the first material 12 and a second component 120 of the second material 22 and a third component 130 of the third material 32. Exemplary configurations 101a, 101b of the filament 100 are described with reference to FIGS. 1A and 1B and FIG. 5A.

The filament 100 of FIG. 1A has a configuration 101a in which the materials and components are arranged having particular forms, locations and dimensions. The first material 12 is different from both the second material 22 and third material 32. The first, second and third components 110, 120 and 130 are arranged about a central longitudinal axis of the filament. The third component 130 is provided as a central core layer, surrounded by middle layer second component 120. The second component 120 is surrounded by the first component 110 arranged as an outer layer.

Thus the second component 120 surrounds or encapsulates the third component 130. The first component 110 surrounds or encapsulates the second component. The first, second and third components are structural components of the filament. The filament 100 comprises a core-sheath filament type structure, the first component 110 defines an outer sheath portion, and the second component 120 defines a middle layer portion and the third component 130 defines an inner core portion.

While FIG. 1 provides a lateral cross-sectional view of a body of the filament 100, it will be appreciated that the filament 100 has an elongated form as shown in FIG. 5A. The first and second components 110, 120, 130 being arranged about a central longitudinal axis (X1-X1) thereof and extending in a longitudinal direction. The components 110, 120 and 130 each has an elongated strand form. Thus each component has an elongated form. In the exemplary arrangement shown both first and second and third components 110, 120, 130 have a generally cylindrical form and extend co-axially along the central longitudinal axis (X-axis) of the filament 100, the second component 120 being arranged to extend radially outwardly about the third component 130. The first component 110 being arranged to extend radially outwardly from the third and second components 130, 120. The filament 100 comprising the first, second and third components also has a generally cylindrical form.

The third component 130 extends radially from a central axis 105 of the filament to a first peripheral surface 131 of the core portion which defines a boundary layer at which the first component is interfaced to the second component. The third component 130 has a first radial extent r1. The second component 120 has an inner peripheral surface 123 at which the second component interfaces the first component, and an outer peripheral surface 121. The second component layer extends radially from inner peripheral surface 123 to outer peripheral surface 121 which defines a boundary layer at which the second component interfaces to the third component. The peripheral surface 121 defines the peripheral boundary at a second radial separation r2 from the central axis 105. The second component 120 has a middle layer depth d2. The first component 110 extends radially from an inner peripheral surface 113 that interfaces the outer peripheral surface 121 of the middle layer to an outer peripheral surface 111 at a third radial separation r3 from the central axis 105. The first component 110 has an outer layer depth d3. The third component 130 is arranged encapsulated within the second component 120. The outer surface 111 of the first portion 110 defines the external peripheral surface of the filament.

The proportions of each of the first and second components 110, 120 and 130, and of the first material 12, second material 22 and first material 32 within the overall volume of the filament 100 can be varied as required. The ratio of the volumes of the first material 12 relative to the second material 22 and third material 32 may be varied between 60:20:20, 20:60:20 and 20:20:60. The proportions may be adjusted depending on the application of the filament 100 as a suture or a component of a multifilament suture.

The structure of the filament and ratios and proportions by volume of the first, second and third materials are varied to control the rate of change or degradation of the suture over time when it is in situ at a surgical site. This allows for the control of the direction and magnitude of forces applied at the surgical site. This also allows for a control of change in strength of the filaments and any single or multi-filament suture over time.

The first, second and third components 110, 120 and 130 are bonded together to form the single composite filament 100. The first and second components 120, 110 are bonded together directly at a bonding interface 113/121. Bonding of the first and second components may be provided by a suitable thermal bonding process, including for example extrusion, spinning or electrospinning.

The second and third components 120, 130 are bonded together directly at a bonding interface 123/131. Bonding of the second and third components may be provided by a suitable thermal bonding process, including for example extrusion, spinning or electrospinning.

The first component 110 in the exemplary arrangement is bonded to the second component 120 at the bonding interface 113/121. At the bonding interface 113/121, the outer peripheral surface 121 of the second component 120 is bonded directly to an inner peripheral surface 113 of the first component 110. The second component 120 in the exemplary arrangement is bonded to the third component 130 at the bonding interface 123/131. At the bonding interface 123/131, the inner peripheral surface 123 of the second component 120 is bonded directly to an outer peripheral surface 131 of the third component 130.

The positive and direct bonding of the components in key in providing a composite filament including the first, second and third materials arranged integrally therein and having the required strength and structural form for the requirement applications in surgery or surgery type.

The first, second and third components 110, 120 and 130 are accordingly bonded together to define an integrally formed filament structure which defines the monofilament 100.

The arrangement of the filament 100 in which the first, second and third materials are bonded as described to form the composite monofilament provides advantage including relating to the uniformity and structural integrity of final construction. The connection between the components of the first material, second and third materials at the bonding interface 113/121 and 123/131 maintains structural uniformity and structural integrity along the length (L1) of the filament. This in turn provides for a uniformity in the forces applied by the filament over the length thereof.

Further, prior to thermal bonding the polymers of the first, second and third materials 112, 122, and 132 may be dried before bonding to remove moisture. This provides that when bonded contaminant levels in the composite filament 100 are limited within the internal molecular structures of each polymer. Contaminants, including for example moisture or H₂O, if present could interfere with the ability of the polymers to bond. The filament 100 thus comprises a monofilament 100 having integrated within a single composite body thereof, the first and second components 110, 120, and 130 having uniformity and structural integrity across the entire body of the filament in a structural configuration. The first component 110, the second component 120 and the third component 130 are structural components of the filament. These components when bonded together form the filament. The filament comprises within the body thereof, portions of the first, second and third materials which have been combined together into a single composite filament structure. In one exemplary arrangement the filament 100 of FIG. 1 is comprised of the first, second and third materials only.

In a further exemplary arrangement the filament 100 may comprise further materials in addition to the first, second and third materials. The filament 100 of FIG. 1B has a form 101b which in comparison with the filament of FIG. 1A further comprises an external coating layer 50. The external coating may for example comprise a collagen coating. Other coating materials could also be used. Further it will be appreciated that while the filament of the exemplary arrangement comprises the first, second and third materials only within the internal body portion, the internal body of the filament being the body portion internal of the outer peripheral surface of the composite filament, in an alternative arrangement other additional materials may be integrated into the internal body portion of the composite filament.

It will be appreciated that while in the example arrangement of FIGS. 1A and 5A the first, second and third components 110, 120 and 130 shown have a cylindrical form (in the longitudinal direction) that other suitable forms may be also used. While in the arrangements of exemplary FIGS. 1A and 1B a center axis of the elongate core 130 is aligned with a center axis 105 of the filament 100 and of the sheaths 120, 110, it will be appreciated that in an alternative arrangement the elongate core component 130 may be positioned not along the center axis of the sheath(s) and not aligned with the center axis of the sheath(s) and filament.

The filament 100 is provided for use as a single filament suture, as one filament of a multi-filament suture 1001 or as one filament of a multi-filament mesh 2000. A multi-filament suture 1001 or mesh 2000 may comprise multiple filaments 100 (or 200, 300). An exemplary multi-filament suture 1001 may comprise multiple filaments 100 in a braided arrangement with other filaments 100 of the same type, or together with one or more different filaments of the same materials or different materials.

In one arrangement of filament 100 the first material 12 is absorbable, and the second materials 22 and 32 are non-absorbable or absorbable.

In another arrangement of filament 100 the first and second materials 12 and 22 are absorbable, and the third material 32 non-absorbable. Filament 100 and a suture or mesh comprising filaments 100 may thus be configured by selection of the materials and the structural components such that in use when the suture or mesh is located at a surgical site, the first material is configured to absorb over a period of time leaving in place the second component comprised of a different absorbable material configured to absorb over a period of time leaving in place a non-absorbable strand or component.

The parameters relating to the absorption of the first material can be controlled by the selection of material and by the dimensions and form of the components comprising the first material, the second material and the third material which together form the composite monofilament 100. Selection of the materials and components allow control over the dynamic changes in the filament over time.

Following on from the above description, a filament 200 of a second type according to an alternative exemplary arrangement of the specification is described with reference to FIG. 2 and FIG. 5B.

Filament 200 similar to the filament 100, and comprises first, second and third materials. Filaments 200 of the second type are described as “islands in the sea” type filaments in which islands of the second and third materials are arranged surrounded by the first material. Various exemplary configurations 201a, 201b, 201c and 201d of the filament 200 are described with reference to FIGS. 2A to 2D.

In the exemplary arrangement of FIG. 2A, the filament 200 comprises components 210, 220 and 230 (also referred to as portions) of a first material, a second material and a third material respectively. As illustrated in the lateral cross-sectional view-the first, second and third materials are provided arranged together in an “islands in the sea” type structural arrangement, in which one or more components 220 (or portions) of the second material 22 and one or more components 230 of the third material 32 are located spaced apart from each of the other components (or portions) of the second and third material. Each of the components 220 and 230 of the second and third materials 22 and 32 is in contact only with the surrounding area of the first material 12 of the first component 210. The first component 210 of the first material 12 is located surrounding the components 220 of the second material 22 and separating them from each other. The first component 210 of the first material 12 is located surrounding the components 230 of the third material 32 and separating them from each other.

Thus, filament 200 is formed from a first portion 210 (or component) of a first material 12 and at least one component 220 of the second material 22. The one or more components 220 (220-1, 220-2 . . . 220n) are encapsulated within the first material 12. Filament 200 further comprises at least one portion 230 of the third material 32. The one or more portions 230 (230-1, 230-2 . . . 230n) of the third material 32 are encapsulated within the first material 212. An external surface 211 of the filament 200 is defined by the outer peripheral surface of the first component 210.

While FIG. 2A to 2D show lateral cross-sectional views only of various configurations 201a, 201b, 201c and 201d of filament 200, it is appreciated that filaments 200 have an elongated filament form overall as shown in FIG. 5B. It will be appreciated that the filament extends longitudinally along and about the central longitudinal axis (X1-X1) thereof and that each component also has an elongated strand form.

The first component 210 forms the main portion of the body of the filament 200. It is arranged radially about a central axis 205 of the filament. The filament 200 has a radius r4. The first component 210 is comprised of the first material 12. An outer peripheral surface 211 of the first component 210 defines the outer surface of the filament. The first component 210 comprises a plurality of internal interface surfaces 213 at which the first component 210 interfaces with the one or more second and third components.

In the exemplary arrangement of FIG. 2A there are four components 220 (220-1, 220-2, 220-3 and 220-4) of the second material 22 and three components 230 (220-1, 220-2, 220-3) of the third material 32 located within the first component 210.

Referring to FIGS. 2B and 5B, filament of configuration 200b is shown. Filament of configuration 200b is an exemplary arrangement of a filament of the second type 200 according to the specification.

In exemplary arrangement of FIG. 2B, the filament 200b comprises a first component 210 of first material 12 and two second components 220 (220-1, 220-2) of second material 22 and two third components 230 (230-1, 230-2) of the third material 32.

Thus each of the plurality of components 210, 220 and 230 comprises a longitudinal strand form L1. In the arrangement each second portion strand 220 has a radial extent r5 or diameter d5, comprising an outer peripheral surface 221 and a longitudinal extent. The strands 220 are arranged spaced apart within the first portion 210. In the exemplary arrangement, each of the portions/strands 220 of the second material are separate from the other portion 220 of the second material. Each portions 220 of the second material is effectively surrounded by, and in contact with the first material 220 of the first component 210 only. The outer peripheral surfaces 221 of the second components 220 bounds or interfaces with an interface surface 213 of the first component.

Each of the plurality of strands of third portions 230 comprises a longitudinal strand form. In the arrangement of each third portion strand 230 has a radial extent r6 and diameter d6 and comprises an outer peripheral surface 231 and a longitudinal extent L1. The strands 230 are arranged spaced apart within the material of the first component 210. In the exemplary arrangement, each of the portions/strands 230 of the third material are separated from the other portions 230 of the third material and portions of the second material. Each portion 230 of the third material is effectively surrounded by, and in contact with the first material 12 of the first component 210 only. The outer peripheral surfaces 231 of the third components bounds or interfaces with an interface surface 213 of the first component.

In the arrangement shown the strands 220, 230 and 210 have a generally cylindrical form, it will be appreciated that other forms are possible.

While in the arrangement 201a of FIG. 2A there are four portions 220 of the second material 22 and three portions of the third material 32 arranged within the body of the filament, it will be appreciated that different numbers of these portions may be provided, as required to provide the controlled degradation of the filament over time. In the arrangement shown the diameter of each of the portions 220, 230 of the second and third materials are the same, it will however be appreciated that different of the portions 220, 230 may be provided having different diameters. The portion 210 of the first material 12 defines the outer or external surface 211 of the filament 200.

As described, above the final filament 200 comprises a monofilament of the components 220 of the second material and 230 of the third material are bonded to the component 210 of the first material to provide a single composite monofilament structure. The filament includes in the body thereof, portions of first, second and third materials components which have been combined together into a single composite filament structure. The filament 200 of the exemplary arrangement of FIG. 2 is comprised of the first, second and third materials only.

The proportions of each of the first, second and third materials within the overall volume of the filament 200 can be varied as required. As described above the ratio of the volumes of the first material relative to the second material and the third material may be varied between 60:20:20; 20:60:20; and 20:20:60. The proportions may be adjusted depending on the application of the filament 200 as a suture or a component of a suture. The first component 210 is bonded to each of one or more second components 220 and the one or more third components to form a single composite filament 200.

As shown, the first component 210 and each of the second components 220 are bonded together directly at each of a plurality of bonding interfaces 213, 221. Similarly the first component and each of the third components 230 are bonded together directly at each of a plurality of bonding interfaces 213, 231. Bonding of the first and the plurality of second and third components 220, 230 may be provided by a suitable bonding process, including for example extrusion, spinning or electrospinning.

At the bonding interface 213,221 the outer peripheral surface 221 of a second component 220 is bonded directly to the surrounding material of the first component 210 at an opposing contact surface 213. At the bonding interface 213,231 the outer peripheral surface 231 of a third component 230 is bonded directly to the surrounding material of the first component 210 at an opposing contact surface 213. The first component 210 is thus bonded to each of the plurality of second components 220 and each of the plurality of third components to define a single composite filament structure. The filament 200 comprises a monofilament 200 having integrated within a single composite body form thereof, the first component 210 and one or more second components 220 and one or more third components 230.

The filament 200 is provided for use as a filament of a suture or a mesh. As described above the first material is absorbable; and the second material is non-absorbable. Thus the filament 200 is configured such that, in use when a suture comprising one or more filaments 200 is located at a surgical site, the first material is configured to absorb over a period of time leaving in place a plurality of separate strands of the second material.

FIG. 5B shows a perspective cutaway view of the filament 200b of FIG. 2B. As shown the filament 200b comprises a central longitudinal axis (X-X) and comprises in a single filament 200b components 210, 220 and 230 of each of the first, second and third materials 12, 22, 32.

The filament 200 is provided for use as a filament of a suture or a mesh. A suture or mesh may comprise multiple filaments 200. An exemplary suture may comprise multiple filaments 200 in a braided arrangement with other filaments 200 of the same type, or together with different filament. As described above the first material 212 is absorbable, and the second material 222 is nonabsorbable and the third material 232 is absorbable or non-absorbable. Filaments 200 and a suture or mesh comprising filaments 200 are thus configured such that in use when the suture or mesh is located at a surgical site, the first material is configured to absorb over a period of time leaving in place the plurality of second components which in the arrangement described are provided as a plurality of strands comprised of the second non-absorbable material. The parameters relating to the absorption of the first material can be controlled by the selection of material and by the dimensions and form of the components comprising the first material and the second material which together form the composite monofilament 200. For example, the selection of a particular first material may allow for control of the time period over which absorption is expected to occur.

In the arrangements of FIGS. 2A and 2B, the filaments 200 are comprised of the first, second and third materials only. It is appreciated that in other alternative arrangements of the specification, the filaments may comprise further materials.

Referring to FIG. 2C, in the example configuration 201c of filament 200, the filament further comprises a coating 50 and components 280 and 290 of materials 82 and 92 respectively. The external coating may for example comprise a collagen coating. Other coating materials could also be used. The components 280 and 290 comprise a fourth 282 and fifth material 292 respectively. The filament 200c comprises the first, second and third materials. In this example, the filament 200c further comprises additionally fourth and fifth materials. The components 280 and 290 may have a spherical or ellipsoid form and be located at a particular defined location (X1, Y1, Z1) of the elongated filament 200b. Alternatively the components 280, 290 may be provided having a strand form similar to the other components of the filament. The components 280 and 290 are bonded with the other components during manufacture to form a single composite filament.

Referring to FIG. 2D a further exemplary arrangement of a filament 200—in a configuration 201d, is described. The filament 200d comprises first, second and third components, 210, 220 and 230 where components of the first, second and third types are comprised of material 42, 62 and 72 respectively.

The structure the filament 200d is similar to the other exemplary arrangements of filament 200 each of the first, second and third components being bonded together to form a single mono-filament. The materials of each of the components of this exemplary arrangement are different from those of filament 200a, 200b, and 200c.

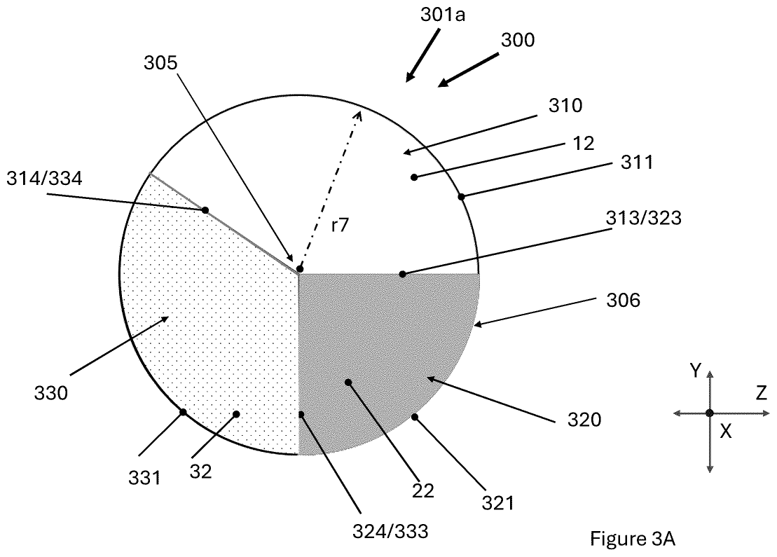

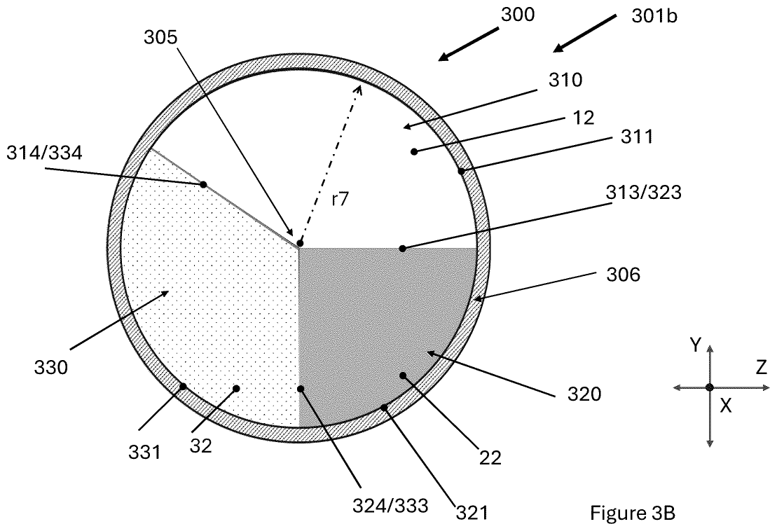

Following on from the above description, a filament 300 according to an alternative exemplary arrangement of the third type of the specification is described with reference to FIGS. 3A and 3B. The filament 300 has a configuration 301a in the exemplary arrangement of FIG. 3A which shows a lateral cross-sectional view of the filament comprising a first material 12, a second material 22, and a third material 32. In particular, the filament 300 comprises a first component 310 of the first material 12 and a second component 320 of the second material 22 and a third component 310 of the third material 32. As illustrated in the lateral cross-sectional view-the first, second and third components 310, 320 and 330 are arranged in first, second and third pie shaped sectors providing a filament 300 having a pie form structural arrangement.

In the cross-section view of the exemplary arrangement of FIG. 3A, the first component 310 subtends an arc of 135 degrees and the first component 310 is provided as a segment of between ¼ and ½ of the cross-sectional area. The second component 320 subtends and arc of 90 degrees and is provided as a segment of ¼ of the area. The third component 330 subtends and arc of 135 degrees. The first, second and third components together define the whole of the circular cross-section.

While FIG. 3 shows a lateral cross-sectional view only of the filament 300, the filament 300 has an elongated filament form overall. It will be appreciated that the filament extends longitudinally along and about the central longitudinal axis thereof. Thus each of first, second and third components comprises a longitudinal strand having a pie shaped form in cross-section.

The outwardly facing surfaces 311 and 321 of each of the first 310 and second 320 components defines a portion of the outer peripheral surface of the filament 300. The second component 320 comprises an internal boundary surface 322. The first component 310 comprises an internal boundary surface 312.

The first component 310, the second component 320, and the third component 330 are structural components of the filament 300. These components when bonded together form the filament 300. The filament comprises in the body thereof, portions of each of the first, second and third materials components which have been combined into a single composite filament structure. The filament 300 of the exemplary arrangement of FIG. 3 is comprised of the first, second and third materials only.

The proportions of each of the first, second and third materials within the overall volume of the filament 300 can be varied as required. The ratio of the volumes of the first second and third materials may be varied within the range of 20:20:60 and 60:20:20 and 20:60:20. In the example of FIG. 3 the ratios are 37.5:25:37.5.

The proportions may be adjusted depending on the application of the filament 300 as a suture or a component of a suture. The proportions and the form and dimensions of the components and materials are adjustable to control the

As described, above the filament 300 comprises a monofilament. The first component 310 of the first material is bonded to the second component 320 of the second material and the third component of the third material to provide a single composite monofilament structure. The first and second components are bonded at a bonding interface 313/323 at which the opposing interface boundary surfaces 313 of the first component and 323 of the second component are bonded directly. The first and third components are bonded at a bonding interface 314/334 at which the opposing interface boundary surfaces 314 of the first component and 324 of the third component are bonded directly. The first and third components are bonded at a bonding interface 314/334 at which the opposing interface boundary surfaces 314 of the first component and 324 of the third component are bonded directly.

Bonding of the first component 310, the second component 320 and the third component 330 may be provided by a suitable bonding process, including for example extrusion, spinning or electrospinning. At the bonding interface 313/323, the boundary surface 313 of the first component is bonded directly to the opposing contact surface 323 of the second component. The first component 310 is thus bonded to the second component 320. The three components are similarly bonded at interfaces 324/333 and 314/344 to define a single composite filament structure. The filament 300 comprises a monofilament 300 having integrated within a single composite body form thereof, the first, second and components 310, 320, 330 of the first, second and third materials.

Each of the components is arranged radially and axially about a central longitudinal axis of the filament. Each component in the exemplary arrangement of FIG. 3A has a radial extent r7, extending form a center point 305 coincident with the central longitudinal axis to an outer peripheral surface 306 of the filament. The outer peripheral surface 306 is made up of the outer peripheral surface portions 311, 321 and 331 of each of the components.

The filament 300 is provided for use as a filament of a suture or a mesh. As described above the first material is absorbable; and the second material is non-absorbable and the third material. Thus the filament 300 is configured such that, in use when a suture comprising one or more filaments 300 is located at a surgical site, the first material is configured to absorb over a period of time leaving in place a strand of the second non-absorbable material.

The parameters relating to the absorption of the first material can be controlled by the selection of material and by the dimensions and form of the components comprising the first material and the second material which together form the composite monofilament 300. For example, the selection of a particular first material may allow for control of the time period over which absorption is expected to occur.

Referring to the exemplary arrangement of FIG. 3B, the filament 300 in a configuration 300b further comprises an outer coating 50 applied to the external peripheral surface of the filament 300. As such the outer coating does not define a structural feature of the filament, but rather a coating applied to at least a portion of the external peripheral surface thereof. The external coating may for example comprise a collagen coating. Other coating materials could also be used.

While in the preferred exemplary arrangements of the drawings, the filaments 300 are comprised of the first second and third materials only, it will be appreciated the filaments 300 or 300a may include further or additional materials similar to components 280 and 290 as described above with reference to filament 200.

The arrangement of the filaments 100, 200, 300 and the other described variations in which the first, second and third materials are bonded as described to form the composite monofilament provides advantage-including relating to the uniformity and structural integrity of final construction. The connection between the components of the first material, the second material and the third material at the bonding interfaces maintains uniformity and structural integrity along the length (L1) of the filament.

Further, prior to thermal bonding the polymers of the first and second material may be dried before bonding to remove moisture. This provides that when bonded contaminant levels in the composite filament are limited within the internal molecular structures of each polymer.

Contaminants, including for example moisture or H2O, if present could interfere with the ability of the polymers to bond. The filaments 200, 300 thus each comprise a monofilament 200, 300 having integrated within a single composite body thereof, the first, second and third components and having uniformity and structural integrity across the entire body of the filament 100, 200, 300. The filaments 100, 200 and 300 as described comprise suture filament which are used in application for forming multi filament sutures or mesh structures for use in medical and surgical applications. The filaments are single composite monofilaments formed from first and second materials also referred to as bicomponent filaments.

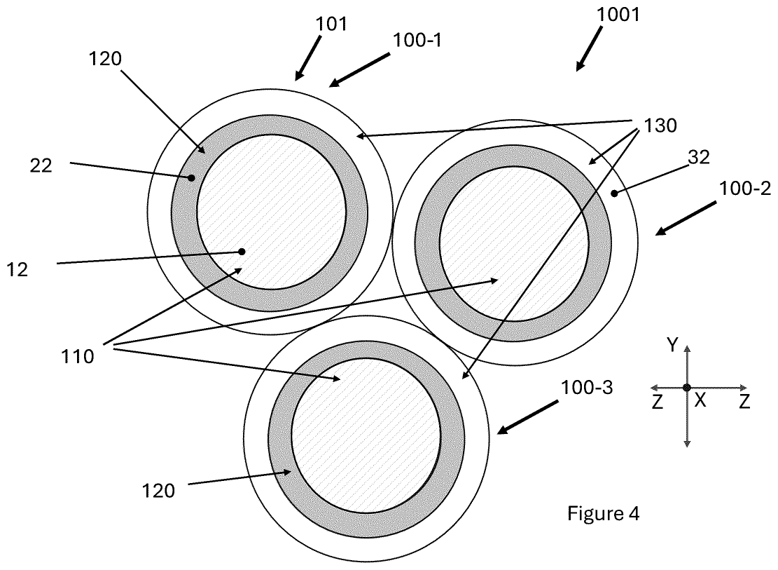

Referring to FIG. 4 multifilament sutures 1000 according to exemplary arrangements of the specification are described. The multifilament suture 1000 shown comprises a plurality of the monofilaments 100 of FIGS. 1 and 5A, the monofilaments are of the first type 101. As described above a multifilament suture 1000 may comprise only monofilaments 100 of the same type. Alternatively a multifilament suture 1000 may comprise one or more monofilaments 100, 200, 300 of the same or different type in combination with another filament of a different material. A multifilament suture may be formed by twisting, braiding or otherwise mechanically combined the filaments thereof to form said multifilament suture 1000. Similarly the filaments 100, 200, 300 may be used to form a mesh 2000.

Advantageously, the arrangements of the disclosure provide dynamic filaments, in which the structure of the filaments 100, 200, 300 which based on the combination of the first, second and third materials changes over time when in location at a surgical site. Advantages of the dynamic filaments and sutures or meshes comprising the dynamic filaments include:

Advantages of filament according to the specification include the following:

Configured to support the third phase of wound healing (inflammatory, fibroblastic and fibrous phases). During the third phase of wound healing, also referred to as the ‘remodeling’ or ‘rebuilding’ phase, the filament has reduced in size, leaving negative space for the body to begin rebuilding at the wounded site with natural, healthy tissue. This is in comparison with other sutures arrangement which often support only the first phase of healing and the early portion of the second phase, resulting in stress shielding from the soft tissue at the wound site.

The self-contained filaments 100, 200, 300 according to the specification provides a sutures configured to maintain the capability of high initial load strength and has the added advantage of maintaining a load strength proportional to the bodily requirements throughout the three-phase healing process.

Advantageously, filaments according to the specification may be configured such that the form of the filament changes dynamically over time such that the properties of forces applied by the filament change dynamically overtime. The properties of the forces include direction and magnitude. The form and rate and rate of change of the filament and the related properties of the forces applied are controlled by the selection of materials and parameters of a filament.

While a coated suture may be provided simply to reduce knot irritation and soft tissue abrasion by utilizing the soft nature of the coating, the arrangements of filaments and suture of the specification comprising the filaments provide advantages and support medical and surgical applications as described above.

Further, the composite filaments 100, 200, 300 provided in accordance with the disclosure-which combine within the single composite filament structure portions of the first and second material have the advantages of providing high levels of uniformity and structural integrity across the lifetime of use of the filament.

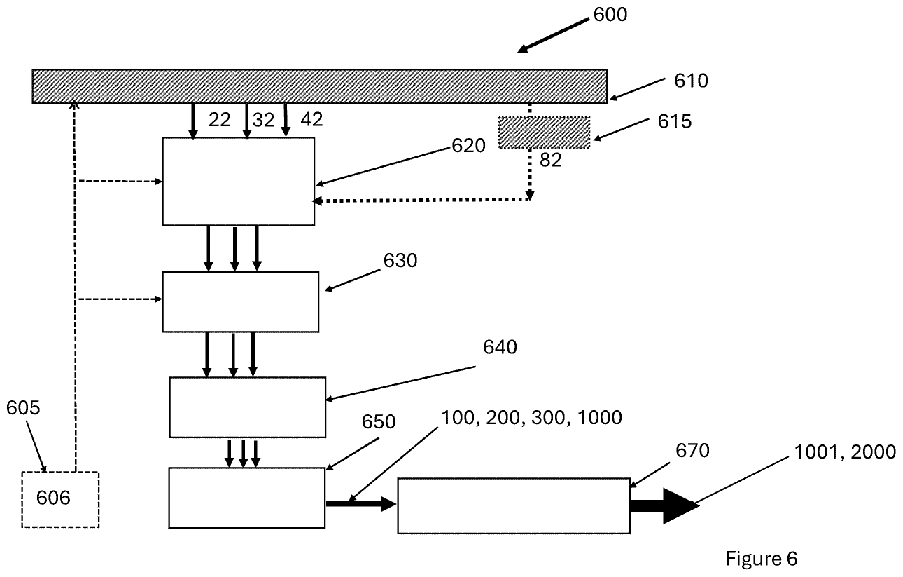

Referring to FIG. 6 an exemplary method 600 for manufacturing a filament according to the specification includes the following:
Selecting 610 first, second and third materials 22, 32, and 42
Selecting 620 the type of filament-from the options 100, 200, 300
Defining 630 the parameters, forms and dimensions of the components of the materials of the filament to be combined in the composite filament, the ratios of the materials. Defining the form and dimensions of the filament.

Pre-treatment 640 of the one or more materials, as required. The pre-treatment may include drying the materials to remove any H₂O and/or removal of any contaminants. The pre-treatment may include a washing of the materials, as required.

Prior to thermal bonding the polymers of the first, second and third materials 12, 22, and 32 may be dried before bonding to remove moisture. This provides that when bonded contaminant levels in the composite filament are limited within the internal molecular structures of each polymer.

Contaminants, including for example moisture or H₂O, if present could interfere with the ability of the polymers to bond.

The filament 100 thus comprises a monofilament 100 having integrated within a single composite body thereof, the first and second and third materials provided in components 110, 120, and 130 arranged across the body of the filament in lateral and longitudinal cross-sections.

Bonding 650 of the components to form a single composite filament 100, 200, 300. The bonding 650 may be thermal bonding, extrusion or any suitable bonding to provide a positive and direct bonding of the materials at the material interfaces within the body of the filament to form a single composite filament of high structural uniformity across the body of the filament in cross-section and continuously over the length thereof.

The bonding 650 further may be provided by spinning the at least three material. The spinning may be melt spinning the at least three materials, electrospinning the at least three materials, or solution spinning the at least three materials.

The parameters relating to the absorption of the first material and any absorbable second or third material can be controlled by the selection of material and by the dimensions and form of the components comprising the first material, the second material and the third material which together form the composite monofilament 100. For example, the selection of a particular first material may allow for control of the time period over which absorption of that material is expected to occur.

Further processing of the filaments may be provided to form 570 a multi-filament suture 1000 or mesh 2000. The forming 570 of a multi-filament suture may comprise braiding of multiple strands of the filament 100, 200,300 to form a multifilament suture 1000. The forming 570 of a multi-filament mesh may comprise braiding of multiple strands of the filament 100, 200,300 to form a multifilament 2000. The forming 570 may comprise using multiple filaments of the same type or of using multiple filaments of different types.

The method 600 may further include receiving inputs 605 relating to the type of surgery for which the filament and suture is to be used, the location of the surgery, the amount and/or location of suture to be deployed. The method then provides that an analysis 606 of the inputs 605 received relating to the type of surgery may be provided for use in selecting the materials of a filament based on the how the suture is required to change dynamically over time.

The analysis 606 of the inputs may also used to guide the form of the filament and internal components there. The analysis 606 includes determining a projected duration of healing for a plurality of phases as a function of time, and related requirements for strength of the suture as a function of time across the projected duration of healing.

Using the analysis 606 of duration and of strength requirements to determine the materials to be used and a ratio of materials of each type making up the overall volume of the filament. In the arrangements of the specification.

Optionally the method 600 may further include selecting 615 one or more further materials to be included in the filament in addition to the first, second and third materials.

The inputs 605 received may further include patient type specific information, such as age, prior medical history. The inputs relating to type of surgery may further include outcome data relating to previous surgical procedures and providing data as to the effectiveness of material combinations and filament types for particular surgical procedures. A machine learning system may be coupled to the system for manufacture of a filament to allow for analysis to support selection of materials and filament manufacture.

Material selection—Assessing the procedure for the intended use of the final suture can influence the selection of materials to include. For example, an ACL repair typically takes longer to heal than a rotator cuff repair and would therefore require the suture to retain more of its initial strength and material properties for a longer period. This would encourage a selection of materials with higher initial tensile strength, a lower degradation rate, or both. It could also influence the desired ratio of resorbable to non-resorbable materials.

The arrangements of the specification advantageously allow for customisation of a filament or a suture according to the surgery or the requirements of the type surgery for which the suture is used.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for anyone of the elements in the claims below or that a single element can be substituted for two or more elements in a claim. Although elements can be described above as acting in certain combinations, and even initially claimed as such, it is to be expressly understood that one or more elements from a claimed combination can, in some cases, be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible considering the above teachings without departing from the following claims.

What is claimed is:

1. A suture filament, comprising:
a first material;
a second material; and
a third material; wherein the first material is absorbable, the second material is absorbable or non-absorbable; and the third material is absorbable or non-absorbable; wherein the materials are bonded together to define a single composite filament.

2. The suture filament of claim 1, the filament having an elongated form wherein the first, second and third materials are arranged extending along a longitudinal axis (X1) of the filament.

3. The suture filament of claim 1, wherein the materials are bonded together wherein a bonding process is selected from the following: a thermal bonding process, an extrusion process, a spinning process and an electro-spinning process.

4. The suture filament of claim 1, wherein the materials are bonded directly at one or more bonding interfaces, wherein the bonding interfaces are located internally within a body portion of the filament, formed at corresponding opposing contact surfaces of the first, second and third materials.

5. The suture filament of claim 1, wherein in a lateral cross-section of the filament, the first material is arranged surrounding the second material, and the second material is arranged surrounding the third material, in a sheath-sheath and core configuration wherein the second material and third material define an inner core component, and the first material defines an outer sheath component of the filament.

6. The suture filament of claim 5, wherein the second material is absorbable, and the third material is non-absorbable.

7. The suture filament of claim 1, comprising a first component of the first material and a plurality of components of the second material and third material, the plurality of components being encapsulated within the first material.

8. The suture filament of claim 7, wherein in a lateral cross-section of the filament, each of a plurality of portions of the second material and third material is arranged spaced apart from other portions of the second material and third material and each being bonded directly to the portion of the first material only.

9. The suture filament of claim 1, wherein in a lateral cross-section of the filament, a first portion of the first material, a second portion of the second material and a third portion of the third material are arranged in pie shaped sectors such that the filament 300 has a pie shaped structural arrangement of the first, second and third materials.

10. The suture filament of claim 1, wherein the filament comprises a monofilament.

11. The suture filament of claim 1, wherein, first, second and third portions of the first, second and third material are arranged bonded together to provide an integrally formed filament structure which defines a single composite filament.

12. The suture filament of claim 1, wherein the first material is absorbable selected from the following: polyglactin, dexon, poliglecaprone, polyglycolic acid (PGA), polydioxanone (PDO), 15 poly-L-lactic acid (PLLA), or any comparable polymer or any combination thereof.

13. The suture filament of claim 1, wherein the second material is non-absorbable selected from the following: polyester, polyethylene terephthalate (PET), ultra-high molecular weight polyethylene (UHMWPE), nylon, or any combination thereof.

14. The suture filament of claim 1, wherein the second material is absorbable selected from the following: polyglactin, dexon, poliglecaprone, polyglycolic acid (PGA), polydioxanone (PDO), 15 poly-L-lactic acid (PLLA), or any comparable polymer or any combination thereof.

15. The suture filament of claim 1, wherein the third material is absorbable or non-absorbable selected from the following: polyester, polyethylene terephthalate (PET), ultra-high molecular weight polyethylene (UHMWPE), nylon, polyglactin, dexon, poliglecaprone, polyglycolic acid (PGA), polydioxanone (PDO), poly-L-lactic acid (PLLA), or any comparable polymer or any combination thereof.

16. The suture filament as claimed in claim 1, wherein ratios of first material to the second material and third materials are adjustable within ranges of 80:10:10, 10:80:10, and 10:10:80; and 70:15:15, 15:70:25 and 15:15:70; and 60:20:20, 20:60:20 and 20:20:60.

17. The suture filament of claim 1, wherein the filament comprises an elongate core made primarily of the third material, wherein the elongate core is bonded to a sheath made primarily of the second material and the sheath of the second material is bonded to an outer sheath made primarily of the first material.

18. The suture filament of claim 1, wherein the filament is configured to degrade dynamically overtime, wherein the degradation overtime is controlled by a selection of materials and parameters, form and dimension of components of the filament.

19. A monofilament suture comprising a suture filament as claimed claim 1.

20. A multifilament suture, comprising: a first filament and at least one other filament as claimed in claim 1, twisted, braided or otherwise mechanically combined to form said multifilament suture, wherein the first and at least one other filament are the same.

21. A multifilament suture, comprising at least one filament of a first type as claimed in claim 1, twisted, braided or otherwise mechanically combined with at least one other filament of a second different type, wherein the filament of the second different type is comprised of materials different from the first and second materials of the filament of the first type.

22. A multifilament suture, comprising:
a first filament comprising a first material, a second material and a third material, wherein the materials are bonded to form a single composite filament; and wherein the first material is absorbable; the second material is non-absorbable; and the third material is absorbable or non-absorbable; and
a second filament comprising a fourth material, a fifth material and a sixth material, wherein the fourth, fifth and sixth materials are bonded to form a single composite filament, and wherein the fourth material is absorbable; the fifth material is non-absorbable; and the sixth material is absorbable or non-absorbable; wherein the first filament and the second filament are braided together.

23. A suture mesh, comprising:
a first filament and one of more other filaments as claimed in claim 1, wherein the first filament is woven, knit, or chemically bonded to at least one other filament, wherein the first and at least one other filament are the same.

24. A suture mesh, comprising:
a first filament as claimed in claim 1, wherein the first filament is woven, knit or chemically bonded to one or more other filaments of different materials.
